(19) United States
(12) Patent Application Publication
(10) Pub. No.: US 2004/0197807 A1
(43) Pub. Date: Oct. 7, 2004

(54) Method for identifying nucleic acid molecules amplified in a polymerase chain reaction

(75) Inventors: Torsten Schulz, Jena (DE); Eugen Ermantraut, Jena (DE); Seigfried Poser, Jena (DE); Thomas Kaiser, Jena (DE); Klaus-Peter Mobius, Zollnitz (DE); Thomas Ullrich, Jena (DE); Karin Adelhelm, Jena (DE)

Correspondence Address: LERNER, DAVID, LITTENBERG,, KRUMHOLZ & MENTLIK, 600 SOUTH AVENUE WEST, WESTFIELD, NJ, 07090, US

(73) Assignee: Clondiag Chip Technologies GmbH, Jena (DE)

(21) Appl. No.: 10/752,122

(22) Filed: Jan. 6, 2004

Related U.S. Application Data
(63) Continuation of application No. PCT/EP02/07539, filed on Jul. 5, 2002.

(30) Foreign Application Priority Data
Jul. 6, 2001 (DE) 101 32 785.4

(51) Int. Cl. C12Q 1/68; C12P 19/34

(52) U.S. Cl. 435/6; 435/91.2

(57) ABSTRACT

Disclosed is a method for qualitative and/or quantitative detection of nucleic acid molecules amplified in an amplification reaction such as PCR. The change of the mass of oligonucleotides in an amplification solution, comprising the mass of target DNA and optionally, the mass of oligonucleotide primers, is determined during the amplification reaction. Also disclosed is a device for conducting the method.

### CROSS-REFERENCE TO RELATED APPLICATIONS

The present application is a continuation of International Application No. PCT/EP02/07539, filed Jul. 5, 2002, which claims priority from German Application No. 101 32 785.4, filed Jul. 6, 2001, all of which are incorporated herein by reference.

### FIELD OF THE INVENTION

The invention relates to a device and to a method for the qualitative and/or quantitative detection of nucleic acid molecules amplified in a polymerase chain reaction.

### BACKGROUND OF THE INVENTION

In the polymerase chain reaction (PCR), a DNA template is exponentially and sequence-specifically amplified by sequential repetition of a temperature regime (see, inter alia, U.S. Pat. No. 4,683,195, U.S. Pat. No. 4,683,202, U.S. Pat. No. 4,965,188, EP 0 502 588; M. J. McPherson, D. D. Hames, G. R. Taylor, PCR 1 and 2: A Practical Approach, IRL Press at Oxford University Press, Oxford (1995)). At the denaturation temperature, the DNA to be amplified is melted to single stranded DNA. At the subsequent lower annealing temperature, the primers bind to both DNA single strands. This is followed by the slightly higher extension temperature, at which both DNA single strands are filled in by an enzyme-catalysed reaction to form double strands. The cycle can be repeated almost as often as desired until the amplified DNA, i.e. the PCR product, is present in analytically utilizable amounts. PCR is one of the most important methods in DNA analysis, and is therefore of great economic significance.

A major problem in PCR is the determination of the number of PCR cycles that is required for amplification of a desired and, if necessary, analytically utilizable amount of nucleic acid. For example, dyes may be used for this purpose which intercalate in double-stranded DNA or attach to double-stranded DNA, thus making the PCR product visible.

F. Lottspeich, H. Zorbas, “Bioanalytik”, Spektrum Akademischer Verlag, Heidelberg (1998) describes, among others things, the dye SYBR Green, which attaches specifically, similarly to an intercalator, and exclusively to double-stranded DNA. On attachment fluorescence of the dye is activated. The dye is added to the PCR. An increase in the measured fluorescence at the extension temperature indicates the synthesis of double-stranded DNA. Furthermore, the temperature at which the fluorescence disappears due to the DNA melting process can be determined in this method by heating the sample to 95° C. after completion of the PCR. In this way, the GC content of a PCR product can be estimated. Finally, this method is also suitable, after calibration, for determining the concentration of the template, i.e. the initial concentration of the nucleic acid to be amplified.

WO 97/29210 and U.S. Pat. No. 5,716,784 describe what is known as a Taqman procedure, which facilitates an in situ-specific and sequence-specific detection of PCR products. The Taqman procedure uses primers carrying two fluorescent dyes in close proximity to each other. In this way the excited dye transfers its “energy” by resonance transfer to the second dye which emits the fluorescence with a red shift. When the primer is incorporated by the Taq polymerase, it loses the second dye due to the endonuclease activity of the polymerase, so that the emitted fluorescence is characterised by a blue shift. The PCR product is detected by this shift in fluorescence. After calibration, this method is also suitable for determination of the template concentration.

Furthermore, what are known as hybridisation probes have been described by Roche Diagnostics GmbH, Roche Molecular Biochemicals, Mannheim, Germany. These types of hybridisation probes are hybridisable oligonucleotides carrying two different fluorescent dyes at their ends that interact by fluorescence energy resonance transfer (FRET). The hybridisation probes are selected in such a way that they hybridise next to each other on the PCR product, so that a red shift of the fluorescence by energy resonance transfer of the two adjoining dyes is initiated. This colour shift is measured in order to detect the PCR product. An important disadvantage of this method is that the consensus sequence of the PCR product has to be known. After calibration, this method is also suitable for determination of the template concentration.

A disadvantage of the above described methods is that the PCR products must be labeled in order to detect the nucleic acid molecules amplified in a polymerase chain reaction. Another disadvantage of the methods of the prior art is that time consuming electrophoretic treatment of the PCR products is usually necessary in order to determine the molecular weight of the PCR products.

There is therefore a need for alternative detection methods for amplified nucleic acid molecules that do not require labelling of the PCR products and that at the same time facilitate detection of the PCR products in situ during the PCR.

### SUMMARY OF THE INVENTION

It is thus an object of the present invention to provide a method with which nucleic acid molecules that have been amplified in a polymerase chain reaction may be detected qualitatively and/or quantitatively, and in which labelling of the PCR products, for example by dye methods or electrophoretic treatment of the PCR products, is not required.

A further object of the present invention is to provide a method with which the molecular weight of nucleic acid molecules that have been amplified in a polymerase chain reaction can be determined without the need for electrophoretic treatment of the PCR products.

It is further an object of the present invention that in situ detection of the PCR products during PCR should be possible, in order to be able to optimise the duration of the PCR with respect to the number of amplification cycles that are sufficient for the desired amount of PCR product.

A further object of the present invention is to provide a method that enables the determination of the initial template concentration.

Further objects can be gathered from the following description.

The objects of the present invention are solved by providing the embodiments characterised in the patent claims.

Surprisingly, it has now been found that nucleic acid molecules that have been amplified in a polymerase chain reaction may be detected qualitatively and/or quantitatively by detecting the change in the mass of oligonucleotides in an amplification solution during a polymerase chain reaction (PCR).

The oligonucleotides present in an amplification solution are the template nucleic acid molecules to be amplified, the amplified nucleic acid molecules, and the oligonucleotide primers used. The template nucleic acid molecules to be amplified and the amplified nucleic acid molecules are hereinafter also covered by the term “target DNA”.

### BRIEF DESCRIPTION OF THE DRAWINGS

FIG. 1: The mass or mass concentration mcₙ of oligonucleotides in the amplification solution is shown in relation to the number of PCR cycles n for two different target lengths (500 base pairs and 200 base pairs).

FIG. 2: White light interference signals are shown in relation to the wave length. The signals are standardised to values between 0 and 1. The shift of the signal due to the change of the loading of the sensor area (121) with the target is apparent.

FIG. 3: The measured value M obtained by white light interferometry as a product of layer thickness difference Ad and refractive index difference An is shown in relation to the PCR cycle number n for two different target lengths (500 base pairs and 200 base pairs).

FIG. 4: Schematic illustration of the device according to the invention in form of an SMO PCR full assembly (1) consisting of a control computer (300), white light interferometer (200), PCR sensor cell (100) and temperature control system (400).

FIG. 5: Schematic illustration of the PCR sensor cell (1) consisting of housing (110), inlet (111), outlet (112), sensor chip (120) and reaction chamber (114).

FIG. 6: Schematic illustration of the PCR sensor cell (1) along section line A-A with affixed heating unit (130).

FIG. 7: Schematic illustration of the PCR sensor cell (1) with light entrance cone (115), seen from above.

FIG. 8: Schematic illustration of the sensor chip (120) with unloaded sensor area (122) and the light path consisting of incident white light (231) of the white light source (201), first order reflection (233) and second order reflection (234). The wave length-dependent phase shift between first order reflection (233) and second order reflection (234) becomes apparent from the Figure.

FIG. 9: Schematic illustration of the sensor chip (120) with loaded sensor area (123). Compared to FIG. 8, the change of the wave length-dependent phase shift in relation to the load becomes apparent. It is to be noted that this phase shift can not be measured directly, since with a length change of approx. 10 nm it is too small. Thus, the reflected light must be spectrally splitted.

### DETAILED DESCRIPTION

Some terms which will be used to describe the present invention are explained below.

Within the context of the present invention, SMO means a sensor system for molecular weight-specific detection of oligonucleotides.

Within the context of the present invention, SMO-PCR is understood to be a sensor system for molecular weight-specific detection of oligonucleotides during the PCR.

Within the context of the present invention, a polymerase chain reaction (PCR) usually comprises approx. 10 to 50 or more PCR cycles, preferably approx. 20 to 40 PCR cycles, especially preferably approx. 30 PCR cycles.

Within the context of the present invention a PCR cycle means a single amplification step of the PCR.

Within the context of the present invention a PCR product means a product resulting from the amplification or multiplication of the nucleic acid molecules to be amplified by the PCR.

Within the context of the present invention an oligonucleotide primer or primer means an oligonucleotide that binds or hybridises to the target DNA, with the synthesis of the antisense strand of the target DNA starting at the binding site during the PCR.

Within the context of the present invention target or target DNA means the DNA that is copied during the PCR.

Within the context of the present invention template or template DNA is understood to mean the DNA that is to be amplified or multiplied by the PCR.

Within the context of the present invention amplification solution is understood to mean a reaction mixture with which the PCR is to be carried out.

Within the context of the present invention the term dNTP means deoxyribonucleoside triphosphate, i.e. the monomer that is needed as the PCR educt.

The terms A, G, C and T stand for adenine, guanine, cytosine and thymine.

Within the context of the present invention, the term oligonucleotide means a polymer consisting of deoxyribonucleoside triphosphates.

Within the context of the present invention sample solution means the liquid to be analysed, containing the nucleic acid molecules to be amplified.

Within the context of the present invention, denaturation temperature means the temperature at which the double-stranded DNA is separated into single strands during the PCR cycle. The denaturation temperature is generally higher than 90° C., preferably approx. 95° C.

Within the context of the present invention, annealing temperature means the temperature at which the primers hybridise to the target. The annealing temperature is usually in the range of from 60° C. to 70° C. and is preferably approx. 65° C.

Within the context of the present invention chain elongation temperature or chain extension temperature means the temperature at which the DNA is synthesized by incorporation of the monomeric components. The extension temperature is generally in the range of from approx. 70° C. to approx. 75° C., and is preferably approx. 72° C.

Within the context of the present invention hybridisation means the association of two single-stranded DNA fragments, thus forming a double strand. The association always takes place to form pairs of A and T, or G and C, respectively.

Within the context of the present invention universal oligonucleotide sensor area (UvOS area) means a surface on which all combinatorially producible oligonucleotides with a defined length are bound with even distribution.

Within the context of the present invention detection phase means the phase in which the determination of the mass change of the oligonucleotides occurs during the PCR.

Within the context of the present invention amplification phase means the phase in which the PCR takes place.

The invention is based on the fact that at each PCR cycle, the mass but not the number of oligonucleotides increases. This will be explained below in an example, starting from the amplification of a single-stranded product. At the beginning of a PCR, for example 10 μmoles oligonucleotide primers and 1 μmole template nucleic acid molecule are present in the amplification solution. In an ideal PCR with an amplification factor of 2, 4 μmoles of the product and, because primers have been used up for amplification, 7 μmoles of primers are present in the solution after two PCR cycles. As the primers mass generally only constitutes approx. 1/10 of the product, the oligonucleotides mass, comprising the mass of target DNA and the mass of oligonucleotide primers, has only increased by approx. 2.4-fold after two cycles in this example. If the primer concentration at the start of the PCR was 100 times greater than the concentration of the template nucleic acid, the total mass of oligonucleotides would increase after two PCR cycles by a factor of 1.03 in this example. The mass increase takes place in each case by the incorporation of deoxynucleoside triphosphate monomers (dNTPs) that are used for the synthesis of the PCR product.

Thus, the present invention relates to a method for qualitative and/or quantitative detection of nucleic acid molecules amplified in a polymerase chain reaction, in which the change of the mass of oligonucleotides in an amplification solution, comprising the mass of target DNA and, optionally, the mass of oligonucleotide primers, during the polymerase chain reaction (PCR) is determined.

An important advantage of the method according to the invention is that, in contrast to all other known methods, labelling of the amplified nucleic acid molecules, for example by dye methods, is not required for detection of the PCR products; but instead, the detection of a successful amplification during the amplification reaction is based only on the determination of the mass of the generated PCR products. As a result, the costs associated with the detection methods known from the art, especially for dyes such as fluorescent dyes, no longer arise in the method according to the invention. Thus, for example, the costs for the fluorescence-labeled primers that carry two fluorescence dyes in close proximity, and that are specifically used in the Taqman method, can be saved.

Another advantage of the method according to the invention is that a polymerase chain reaction in which labelling methods, for example in the form of dye methods for the detection of the amplified nucleic acid molecules, are no longer needed, is much more robust, i.e. there is less risk of termination of the reaction and/or a higher amplification factor.

It is obvious for the expert that the detection method according to the invention is not limited to the use in PCR reactions. Rather, the invention may be used for other amplification reactions as well. Examples are the so-called IDA reaction (see DE 197 41 714), the ligase chain reaction (LCR method), the P&LCR, the 3SR reaction (self-sustained sequence replication), the NASBA method (nucleic acid sequence based amplification) and the SDA reaction (strand displacement amplification). It is obvious that the detection method according to the invention is suitable for all reactions in the course of which nucleic acid molecules are amplified, i.e. the change of the mass of nucleic acids allows a statement on the success of the amplification.

Preferably, the change in the mass of oligonucleotides during the PCR is determined by determining the mass of oligonucleotides present at one PCR cycle at at least two PCR cycles. Especially preferably, the mass of oligonucleotides present at a PCR cycle is determined at each PCR cycle. Alternatively, the mass of oligonucleotides in the amplification solution or the PCR reaction solution may also be determined at every second cycle or at every third cycle or at any other interval. In order to determine a change in the mass of oligonucleotides during the PCR, it is however necessary to determine the mass of oligonucleotides at at least two cycles of the PCR.

In a preferred embodiment of the method according to the invention, the increase in the mass of amplified nucleic acid molecules is specifically determined, without considering the mass of oligonucleotide primers during the PCR. However, as an alternative it is also advantageous to determine the change in the total mass of oligonucleotides, i.e. the mass of DNA that is duplicated during the PCR and the mass of oligonucleotide primers, during the PCR.

The mass of oligonucleotides present at a PCR cycle is preferably determined by determining the mass of oligonucleotides binding to a surface. Especially preferably, a surface is used in the method according to the invention that binds PCR product and primers with almost the same affinity. However, it may also be preferred to use a surface that preferably binds the target DNA. In both alternatives it is advantageous that the deoxyribonucleoside triphosphates which are used as monomeric components in the PCR do not bind to the surface. Furthermore it is preferred that the binding of the oligonucleotides to the surface is reversible.

In an especially preferred embodiment of the method according to the invention, the determination of the mass change of oligonucleotides takes place in situ, i.e. in the reaction chamber in which the PCR takes place as well. In this way, the number of PCR cycles that are required for a desired amount of target DNA may be determined online. When, for example, the production of the desired amount of target DNA is detected, the PCR may be stopped automatically. Thus, the number of cycles of the PCR may be limited by the method according to the invention to the number of cycles that are sufficient for the amount of target DNA. This in situ optimisation of the reaction time saves time and money.

In a further preferred embodiment, binding sites that are present on the surface after each amplification step are saturated with an oligonucleotide. Since the mass increases in each PCR cycle, where the mole number of oligonucleotides remains unchanged, the mass bound to the surface also increases in each PCR cycle.

In the method according to the invention, the change in mass of oligonucleotides during the PCR is determined preferably using a mass sensitive detector. As a mass sensitive detector, a transducer may be used for example, which transforms a mass change on a surface into an electrical or optical signal. Within the context of the present invention generally those methods that react sensitively to a mass change on a surface film may be used as detection methods. However, it is not necessary that the measured quantity represents the mass change itself. Thus, all mass-dependent measured values, such as refractive index, change in density, change in layer thickness, and compressibility as well as combinations thereof, may also be employed to determine the change in the mass of oligonucleotides in the present method.

The following detectors or transducers are especially preferably used in the method according to the invention:

Surface wave sensors or surface acoustic wave (SAW) sensors using a feedback mechanism to measure the self-oscillation of the sensor surface which depends strongly on its mass. (N. Barie, M. Rapp, H. J. Ache, UV crosslinked polysiloxanes as new coating materials for SAW devices with high long-term stability, Sensors and Actuators B, 46(1998) S. 97-103).

Microbalances based on an oscillating crystal that changes its frequency based on the surface load (G. Schwedt; Analytische Chemie: Grundlagen, Methoden und Praxis; Stuttgart [among others]: Thieme, 1995).

Sensors based on quartz crystal microbalance dissipation, i.e. a refinement of the microbalance method that in addition uses the attenuation of the quartz oscillation (Q-Sense, Gothenburg, Sweden).

Detectors based on interferometric methods in which refractive index and changes of layer thickness are measured.

An especially preferred interferometric method within the scope of the invention is the white light interferometry (W. Nebe, Analytische Interferometrie, Leipzig: Akad. Verl.-Ges., 1970) which, in the case of analytical measurements of changes of layer thicknesses, is also named RIFS (reflectometric interference spectroscopy) (G. Gauglitz, A. Brecht, G. Kraus, W. Nahm; Chemical and biochemical sensors based on interferometry at thin (multi-)layers; Sensors and Actuators B, 11 (1993) 21-27).

Detectors for determination of surface plasmon resonance.

In surface plasmon resonance spectroscopy (SPR), the angle dependency of the reflection intensity, e.g. at the interface system of a glass-gold-sensor surface-amplification solution, is measured (I. Stemmler, A. Brecht, G. Gauglitz; Compact SPR-transducers with spectral readout for biosensing applications; Sens. Actuators B 54, 98-105 (1999)). The plasmon resonance frequency is highly dependent on the density of the sensor area. The change in plasmon resonance frequency is accompanied by a change of the reflection intensity.

In a further preferred embodiment, the method according to the invention comprises the following steps:

a) Denaturing of DNA double strands by setting a suitable temperature (denaturation temperature);

b) Annealing of the oligonucleotide primers to the nucleic acid molecules to be amplified by setting a suitable temperature (annealing temperature);

c) DNA synthesis or chain elongation at a suitable temperature (chain elongation temperature);

d) Optionally, repeating steps a) to c), whereby the determination of the mass of oligonucleotides present at a PCR cycle can be determined after step b) and/or after step c).

In PCR, conventionally three temperatures are run for each PCR cycle. The oligonucleotides bound to the surface preferably detach from the surface at the highest temperature, i.e. at the denaturation temperature. A preferred value for the denaturation temperature is 95° C. Thus, a measured value may be determined at this denaturation temperature, which serves as zero value or reference value for the mass of oligonucleotides determined at the respective PCR cycle.

At the temperature following the PCR cycle, i.e. the annealing temperature of for example approx. 65° C., the oligonucleotides are adsorbed again completely by the surface, for example the sensor area. In an embodiment of the method according to the invention, the mass of oligonucleotides present at a PCR cycle is therefore determined at the annealing temperature.

In order to increase the sensitivity of the method according to the invention, it may furthermore be advantageous to decrease the temperature below the annealing temperature, so that the mass of oligonucleotides present at a PCR cycle is determined preferentially at a temperature below the annealing temperature. For example, the determination of mass may be performed at a temperature in the range of from 25° C. to 50° C., and preferably in the range of from 30° C. to 40° C.

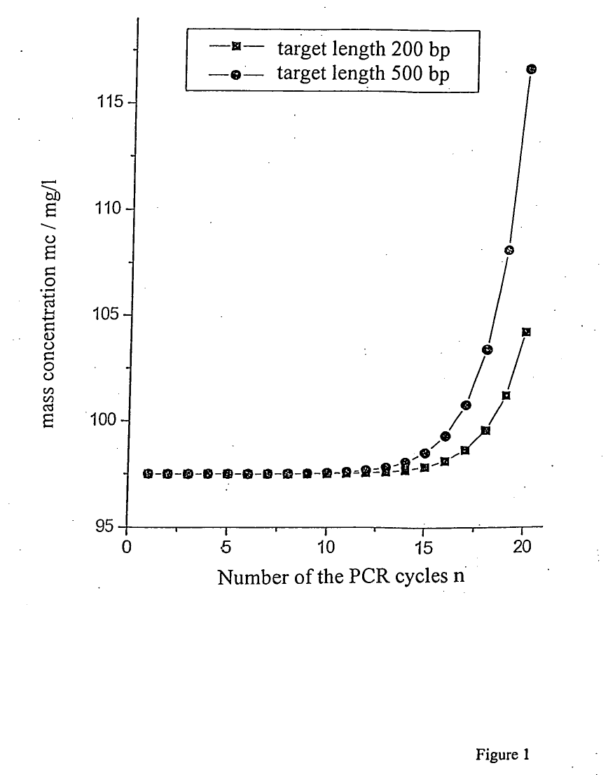

If the zero value or reference value that has been determined at the denaturation temperature is subtracted from the measured value obtained at or below the annealing temperature, an interference-free measurement result is obtained from which fluctuations and drift are eliminated. If suitable mass-sensitive measurement methods are used, this result is proportional to the mass mcₙ of oligonucleotides after n cycles. The result of such a detection method is shown in FIG. 1.

In an especially preferred embodiment of the method according to the invention, the molecular weight of the amplified nucleic acid molecules and/or the amplification factor of the polymerase chain reaction are determined from the change in the mass of oligonucleotides during the PCR.

The molecular weight of the amplified nucleic acid molecules and/or the amplification factor of the PCR are determined according to the equations given below. In these equations, M represents the measurement result which is for example obtained in the detection of mass change by white light interferometry from the product of the refractive index difference Δn and the change in layer thickness Δd. cpₙ represents the primer concentration after n cycles, whereas ctₙ stands for the target concentration after n cycles. n represents the number of PCR cycles, and v indicates the amplification factor of the PCR. mcₙ represents the mass of oligonucleotides after n cycles. lp and lt stand for the length of the primers, or the lengths of the target and PCR product, of the PCR in numbers of bases, whereas MB represents the average mole weight of the four bases which can be set to 325 g/mole. All of the following derivations and concentrations refer to single-stranded DNA.

The target concentration after 0 steps is: ct₀=ct₀*v⁰

In an ideal PCR, the amplification factor is v=2. This means that with every step, i.e. every PCR cycle, the target concentration is doubled.

After the first step, the target concentration is ct₁=ct₀*v, after the second step ct₂=ct₁*v=ct₀*V², and after the nth step ct₁=ct₀*vⁿ.

At each step in which target is generated, primer is depleted, i.e. one primer single strand for each target single strand. Therefore, the primer concentration after the first step is: cp₁=cp₀−ct₀, after the second step: cp₂=cp₁−ct₁=cp₀−ct₀−ct₁=cp₀−ct₀−ct₀*v, after the third step: cp₃=cp₂−ct₂=cp₀−ct₀*(1−v−v²), and after the nth step: cpₙ=cp₀−ct₀*(1−v−v−v³ . . . vⁿ)

The term in parenthesis represents a geometrical sequence. Therefore, cpₙ becomes:

cpₙ=cp₀−(ct₀*(vⁿ−1)/(v−1))

The target length lt, which after multiplication with the average molecular weight of the bases yields the molecular weight of the target DNA, and the amplification factor v may be determined as follows:

The mass concentration mcₙ of oligonucleotides is obtained by multiplying the primer concentrations with the primer length lp and the target concentrations with the target length it, and by multiplying the result with the average mole weight of the bases MB:

mcₙ=MB*cpₙ*lp+MB*ctₙ*lt

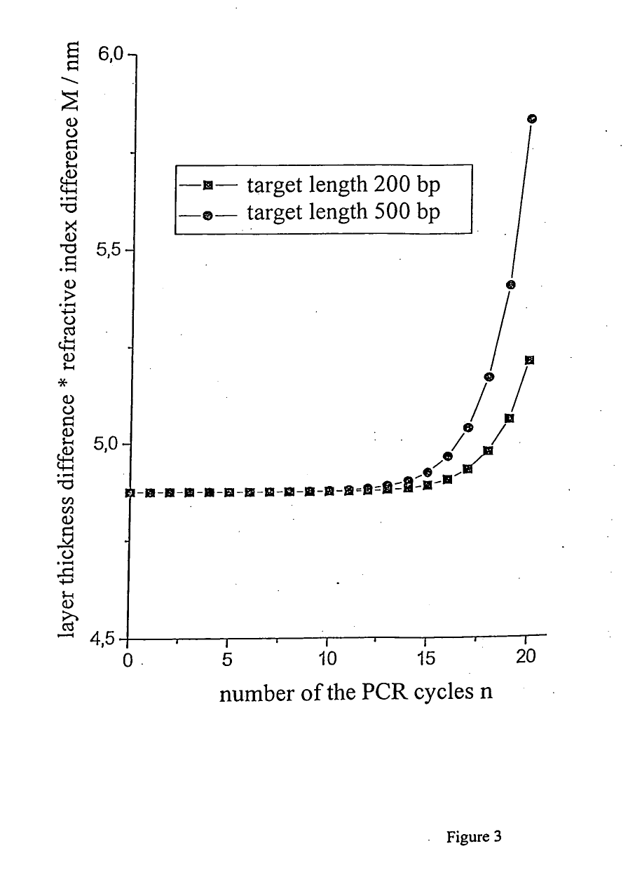

The assumption is made here that for the first PCR steps (in FIG. 3 up to approx. step 20), MB*cpₙ*lp remains almost constant, since the primer consumption relative to the total amount of primer in the solution remains constant during the first PCR steps.

A constant K=MB*cpₙ*lp can therefore be introduced.

The measurement signal Mₙ is linearly dependent on the total mass concentration through the system constant E: Mₙ=E*mcₙ

In the first steps, the signal is generated only by the primers, as almost no target has yet been produced compared to the primer concentration: In this case, Mₛₜₐᵣₜ corresponds to E*MB*cpₙ*lp=K*E

The total measurement signal is obtained from the sum of Mₛₜₐᵣₜ and M′ₙ. M′ₙ=E*MB*ctₙ*lt=E*MB*ct₀*vⁿ*lt.

Since only v and lt are of interest, Mₛₜₐᵣₜ is subtracted from Mₙ:

M′ₙ=Mₙ−Mₛₜₐᵣₜ

Since as a second measured value, besides a measure for the mass of oligonucleotides only the number of cycles or number of steps n is available, and between M′ₙ and n, before a saturation phase is achieved, an exponential correlation exists, in order to establish it and v the measured values M′ₙ are logarithmised in order to then be able to perform a linear regression:

log(M′ₙ)=a*n+b=log(v)*n+log(E*MB*lt*ct₀)

a and b are obtained by linear regression, and then, by transformation, the sought values target length it and amplification factor v.

The change in the mass concentrations depending on the number of PCR cycles for a template which is 500 base pairs long and 200 base pairs long are depicted in FIG. 1.

The advantage of determining the molecular weight of the PCR products in the above described way is that no time-consuming electrophoretic treatment of the PCR products is necessary after the PCR in order to determine the molecular weight.

In a further preferred embodiment of the method according to the invention, in order to determine the initial concentration of the template DNA a number of PCRs with various given initial concentrations of the same nucleic acid molecules, i.e. the same template molecules, is carried out for calibration. Preferably, the PCR cycle in which the amplification-induced change of the mass of oligonucleotides becomes measurable is used as a measure for the initial concentration of the template molecules. Since the change in mass of oligonucleotides during the PCR reaction represents a sigmoid function, comprising an exponential increase and a saturation phase, this initial cycle ns may be established by, for example, determining the inflection point of the sigmoid function, and by applying a tangent to the function through this inflection point. The intercept of this tangent at the x-axis (see FIG. 1) yields the start cycle ns. The inflection point is obtained by determination of the maximum of the first derivation of the sigmoid function.

Alternatively, the start cycle ns may also be established by determining the vertex of the sigmoid function, i.e. the maxima of the second derivation of the sigmoid function, and connecting them with a straight line. Again, the point where this straight line intersects with the x-axis represents the start cycle nₛ.

Thus, the start cycle nₛ that has been determined by the above described manner may be a real number.

In the same way, the start cycle nₛ for the sample with unknown concentration is determined. By regression and interpolation calculations, the initial concentration ct₀ of the template DNA is obtained.

In a further preferred embodiment of the present invention, the temperature at which the oligonucleotides detach from the surface will be determined after completion of the PCR. In this way, information about the specific nature of the PCR products, especially the GC content of the amplified nucleic acid molecules, may be obtained. For example, the temperature may be successively increased after completion of the PCR, and the temperature at which the olignucleotides detach from oligonucleotide probes on the surface may thus be measured.

In a further aspect of the present invention, a device for the qualitative and/or quantitative detection of nucleic acid molecules that are amplified in a polymerase chain reaction is provided.

The device according to the invention comprises a reaction chamber in which the PCR is carried out; a unit for controlling the temperature; a surface that is suitable for the binding of oligonucleotides; as well as a detector for determining the mass of oligonucleotides bound to the surface.

Within the context of the present invention binding or attachment to a surface means any possible kind of interaction of the oligonucleotides with a surface that is suitable for mass-sensitive determination of the binding on the surface.

Preferably, the surface is located on the detector for determining the mass of the oligonucleotides bound to the surface. In this embodiment, the surface will also be designated as sensor area within the context of the present invention.

Basically almost any surface type is suited as surface or sensor area. As described in V. Chan et al., Effect of Hydrophobicity and Electrostatics on Adsorption and Surface Diffusion of DNA Oligonucleotides at Liquid/Solid Interfaces, Journal of Colloid and Interface Science, (1998) 203, 197-207, the binding or adsorption of oligonucleotides to a surface is almost always unspecific.

In a preferred embodiment, the surface binds oligonucleotides unspecifically, i.e. regardless of whether oligonucleotide primers or usually considerably longer template DNA or target DNA are meant. Furthermore it is preferred that the surface binds the oligonculeotide primers and template DNA or target DNA with similar affinity.

In an alternative embodiment of the present invention, the binding of amplified nucleic acid molecules is preferred compared to the binding of oligonucleotide primers to the surface. In this embodiment, a greater change in the mass of oligonucleotides is observed per PCR cycle, since the effect of the primer concentration is less, or, if the oligonucleotide primers do not bind to the surface, the mass of the oligonucleotide primers is not detected.

Furthermore it is preferred that the surface does not bind deoxyribonucleoside triphosphates.

Furthermore, in a preferred embodiment of the device according to the invention, binding sites for the oligonucleotides are evenly distributed on the surface. In this way it is ensured that all oligonucleotides bind to the surface with the same degree of probability.

In a further preferred embodiment of the device according to the invention, the number of binding sites for the oligonucleotides on the surface exceeds the number of oligonucleotides. This has the advantage that all oligonucleotides can bind to the surface, and thus comparable mass values are obtained per PCR cycle.

A particularly advantageous embodiment of the device according to the invention has been shown to be a surface, where the binding sites on the surface are attached oligonucleotide probes which are suitable for hybridisation with the nucleic acid molecules to be amplified, i.e. with the target and template nucleotide sequences, and/or with the oligonucleotide primers.

Here it is particularly preferred that all oligonucleotide probes have the same length.

Furthermore it is preferred that the oligonucleotide probes are produced combinatorially on the surface. A surface or sensor area, on which all combinatorially producible oligonucleotides of a given length are bound with even distribution, is hereinafter called universal oligo sensor (UvOS) area. In order to produce such a UvOS area in the case of a glass chip as a support for the sensor area, for example, the glass surface is silanized with

-glycidoxypropyltrimethoxysilane (GOPS) (see U. Maskos, E. M. Southern, Nucleic Acid Research (1992), 20:1679), the epoxy is opened and esterified with pentaethylene glycol. Then, using phosphoramidite chemistry (M. J. Gait, Oligonucleotide Synthesis: A Practical Approach; IRL Press at Oxford University Press, 1990), surface-bound oligonucleotides are synthesized on the surface; all four bases (A, G, C and T) being simultaneously coupled with each synthesis step. In this way, all possible 2-mers are obtained after the second step, i.e. 16 different oligonucleotides, in even distribution on the surface. After the third step, all possible 3-mers (4³) are obtained, and after the nth step, all possible n-mers (4ⁿ) are obtained on the surface or sensor area. In this way, a hybridisable probe is created on the UvOS area for every imaginable oligonucleotide. Such a method is called combinatorial synthesis.

Every oligonucleotide present in the amplification solution is able to bind or hybridise to such a UvOS area. A UvOS area as the sensor area thus fulfils all requirements for qualitative and/or quantitative detection of nucleic acid molecules amplified in a PCR by determining the mass of the oligonucleotides that are bound to the surface.

With such UvOS areas, the hybridisation temperature is crucial. The lower the temperature, the better the oligonucleotides bind to the surface. In contrast, at higher temperatures, for example at a denaturation temperature of approx. 95° C., any DNA-DNA interaction is reversibly melted during the PCR. Thus, a zero value or reference value for determining the mass change at each PCR cycle can be measured at the denaturation temperature. For determining the mass of the oligonucleotides attached to the surface it may be advantageous to decrease the temperature below the annealing temperature in order to increase the sensitivity of the detection.

Furthermore, a feature of preferred surfaces or sensor areas is that they bind longer DNA molecules better than shorter ones, i.e. template DNA molecules or target DNA molecules are bound better than the shorter oligonucleotide primers. In this way, the signal of the detection of the amplification of target DNA is improved through the mass change.

In order to keep a change of the primer and target concentrations during annealing by binding to the surface or sensor area as low as possible, it is also preferred that the ratio of sensor surface to reaction chamber volume be kept as low as possible. Preferred values for the ratio of surface to chamber volume are in the range of 10⁻¹ mm²/μl to 10⁻³ mm²/μl, particularly preferably 5×10⁻² mm² to 5×10⁻³ mm²/μl, and most preferably approx. 0.02 mm²/μl.

Preferred detectors for determining the mass of the oligonucleotides that are bound to the surface have already been described above, and are preferably selected from the group consisting of a white light interferometer, a surface wave sensor, a microbalance, a quartz crystal microbalance dissipation sensor as well as a sensor for detection of surface plasmon resonance.

If, for example, the determination of the mass is carried out using a white light interferometer, it may be advantageous if the surface of the device according to the invention is of a thickness suitable for interferometry, preferably in the range of from 300 nm to 700 nm, particularly preferably in the range of from 400 nm to 600 nm, and most preferably approx. 500 nm.

In a further preferred embodiment, the device additionally comprises a control computer which records the measured data and/or controls temperature regulation as well as the detector.

Furthermore it is preferred that the device according to the invention additionally comprises an inlet for the samples to be amplified or an outlet for the amplified samples.

A further aspect of the present invention is the use of the above described device according to the invention for carrying out the method according to the invention.

The present invention thus provides a sensor system for molecular weight-specific detection of oligonucleotides that ensures the mass-specific detection of oligonucleotides. Particularly advantageous is the use of the device according to the invention for specific in situ detection of PCR products. Because it is possible to measure the PCR product yield as well as the base length and thus the molecular weight of the PCR products using the above described method according to the invention and/or the device according to the invention, usual time-consuming analysis steps such as electrophoretic treatment of the products in which DNA is separated by size by migration in a gel matrix, are no longer necessary. By availing of the possibility of online detection, the number of cycles can, using the method according to the invention and/or the device according to the invention, be restricted to the number of cycles necessary for a desired amount of PCR product.

The present invention is described by the following example which is however not to be interpreted as limiting the scope of the invention.

### EXAMPLE

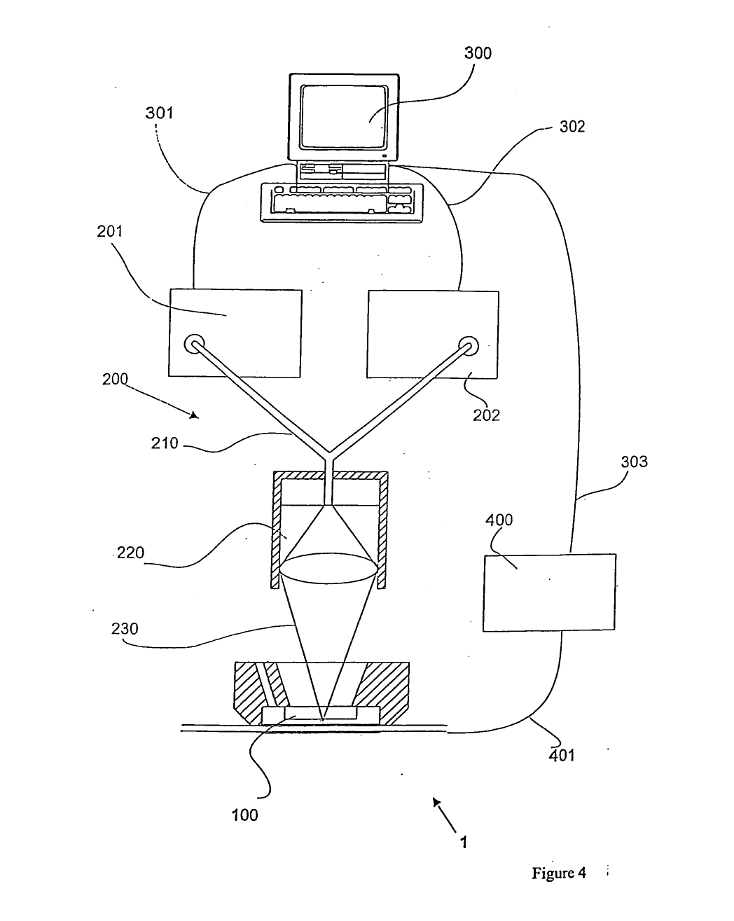

The device according to the invention, in this example designated as SMO-PCR (1), comprises here a white light interferometer (200) with which the change of the layers in the sensor surface (121) of the PCR sensor cell (100) are measured (see FIG. 4). A control computer (300) records the measured values via the control line (302). In addition, the control computer controls and monitors, via the control lines (301, 303), the temperature control system (400) as well as the white light source (201).

Assembly of the PCR Sensor Cell (100)

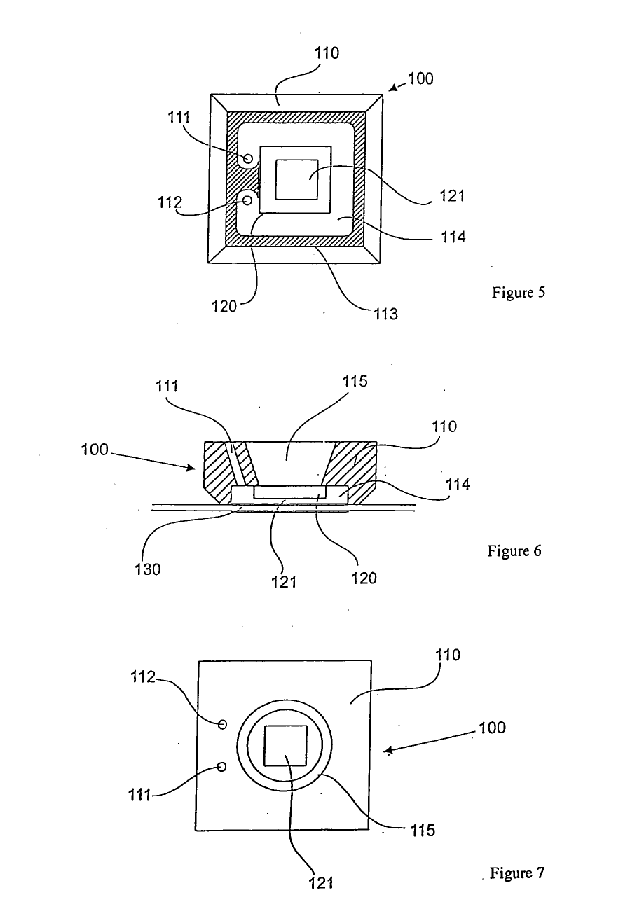

WO 01/02094 describes a miniature PCR chamber that may be used by insertion of a sensor chip (1) according to the present invention instead of the chip used in WO 01/02094 in the sense of the sensor system according to the invention (see FIG. 5). In this respect explicit reference is herewith made to the disclosure of WO 01/02094.

The device according to the invention or the PCR sensor cell (100) consists of a housing (110) in which a chamber (114) is located which may be emptied of, and filled with, sample solution through an outlet (112) and an inlet (111). The chamber (114) has a light entrance cone (115) to measure the interactions at the sensor surface (121) with a white light interferometer (200). The light entrance cone (115) is closed by the sensor chip (120). The underneath side of the chamber (114) is glued to a heating unit (130) and covered. With the heating unit (130), the sample solution can be heated to an exact temperature. A heating unit is described in WO 01/02094. The behaviour of the fluid as well as the reliable wetting of the sensor area (121) are also described in WO 01/02094.

Assembly of the White Light Interferometer (200)

The white light interferometer (200), e.g. the one commercially available from “Ingenieurbüro für Angewandte Spektrometrie”, Röntgenstraβe 33, D-73431 Aalen, Germany, consists of a white light source (201), whose light passes an optical waveguide (210) to reach a focussing front objective or ancillary objective (220). The focussing front objective (220) focuses the light onto the sensor area (121). The light beams (230) reflected from the sensor area (121) are coupled into the two-armed Y optical waveguide (210) by the focusing front lens (220). The reflected light enters the diode array spectrometer (202) and is analysed by measuring the intensity of the reflected light depending on the wavelength.

Sensor Chip (120)

The sensor chip (120) consists of glass onto which a three-dimensional SiO₂ network serving as sensor area (121) has been applied by sol-gel technique (C. J. Brinker, G. W. Scherer, Sol-gel science: The physics and chemistry of sol-gel processing; Boston [among others]: Academic Press, 1990). The sol-gel has a thickness of approx. 500 nm. It is modified with glycidoxypropyl groups to which oligonucleotides are synthesized via pentaethylene glycol. The oligonucleotide synthesis is carried out combinatorially. Using this production method, a UvOS surface is obtained that, having a strength of 500 nm, is rather thick and therefore suitable for white light interferometry. The refractive index is approx. 1.4.

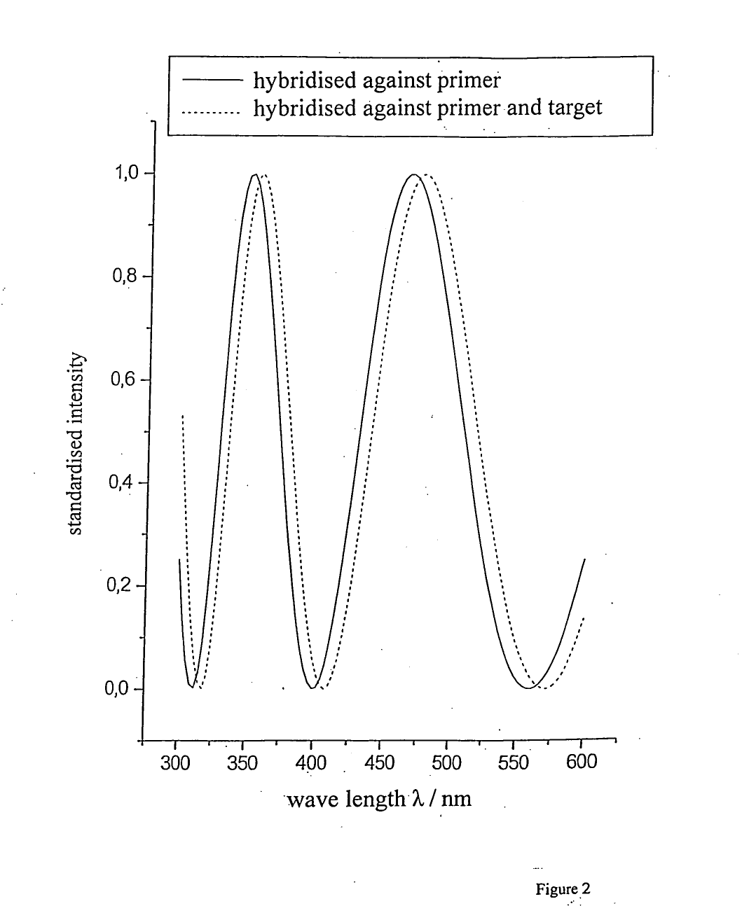

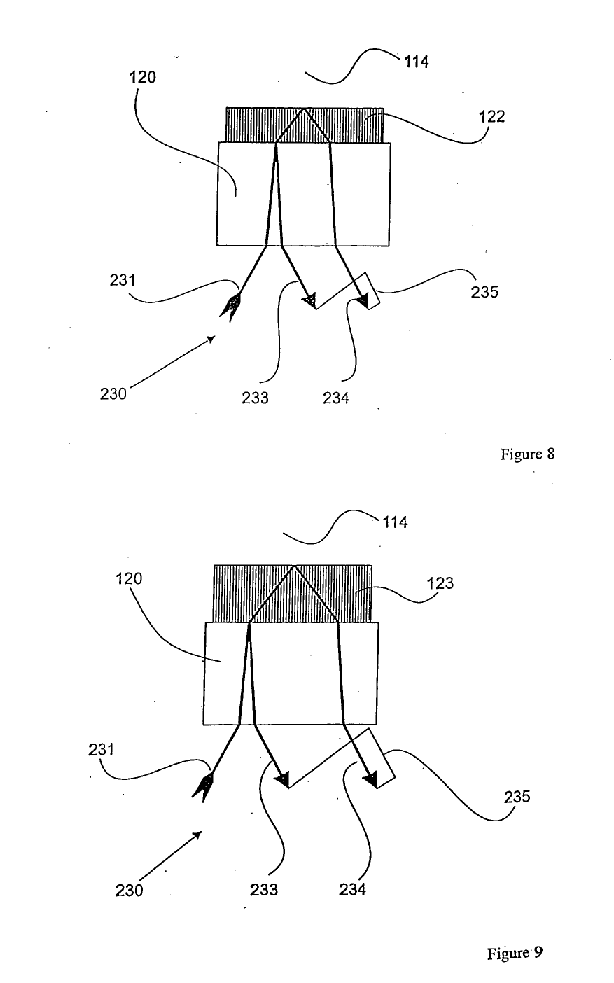

By hybridisation of oligonucleotides, the product of refractive index and layer thickness changes by approx. 10 nm. This change is depicted schematically in FIGS. 8 and 9. Also, the optical path of the white light interferometer (200) is outlined. The incident white light beam (231) is reflected at the interface of sensor chip (120) and sensor area (121) as well as at the interface of sensor area (121) and chamber (114). The first (233) and second (234) order reflections demonstrate a slight phase shift (235) when exiting the sensor chip (120). The thickness of the sensor area (121) generally increases by binding (hybridisation) of oligonucleotides. The phase difference (235) thereby increases during the transition of the unloaded sensor area (122) (see FIG. 8) to the loaded sensor area (123) (see FIG. 9). However, the change of the layer thickness is too small to make a direct measurement of the phase difference by interferometry. The emergent light is thus spectrally splitted by a diode array spectrometer (202), and the light intensity is measured against the wave length (see FIG. 2).

White Light Interferometry Signal

The interference signal demonstrates destructive and constructive interferences depending on the wave length (see FIG. 2). From the shift of two spectra, the product of layer thickness change Ad and refractive index difference An of the surface area (121) may be determined from one measurement to the next (peak to peak distance). The measurement is carried out first at the denaturation temperature of 95° C. and then at the annealing temperature of 65° C. Then, the average values of the distances of the maxima and minima from the white light interferometry spectra are determined. Thus, the product of layer thickness change Ad and refractive index difference An between the unloaded sensor area at 95° C. and the loaded sensor area at 65° C. is obtained for each PCR cycle as a measured value. A corresponding example is shown in FIG. 3.

Data Evaluation (300)

A control computer (300) uses a software program for driving the white light source (201), the temperature control system (400) for the PCR, and for data read-out of the measurement of the diode array spectrometer (202). A computer program calculates the measured value M for each PCR cycle from the measured data of the diode array spectrometer (202) (see FIG. 3). From the change of M with each PCR cycle, the amplification of each PCR cycle may be determined. A strong increase in the measured value M indicates a successful amplification. The measurement can be stopped. From the obtained measured value pairs, the length it and the amplification factor v of the PCR may be determined. In order to do this, the average value of all measurements Mₛₜₐᵣₜ at which no amplification has been detected yet, is subtracted from the remaining measurements Mₙ:

M′ₙ=Mₙ−Mₛₜₐᵣₜ

Measured values M′ₙ are obtained. The measured value pairs thus obtained are logarithmised, and then the coefficients from the following equation are determined by linear regression:

log(M′ₙ)=a·n+b

From this, the length it of the PCR products and the amplification factor v may be calculated as follows:

v=exp (a),

lt=exp(b)/E·ct₀·MB

wherein E is the system constant of the measuring apparatus. For the curves depicted in FIG. 3, values of 170 (theoretically 200) or 470 bp (theoretically 500) are obtained for the target length lt.

The initial concentration of the target ct₀ is obtained by calibration measurement. Several polymerase chain reactions with the same template of different, but known, initial concentrations are analysed. For each PCR, the start cycle ns is determined, from which on the amplification becomes measurable. Since the start cycle is obtained by extrapolation, nₛ is a real and not a natural number. The start cycle of the sample with unknown concentration is determined in the same way. By regression and interpolation calculation, this yields the initial concentration ct₀.

1. A method for qualitative and/or quantitative detection of nucleic acid molecules amplified in an amplification reaction, comprising determining a change in mass of oligonucleotides comprising target DNA, wherein said determining is conducted during the amplification reaction.

2. The method of claim 1, wherein the amplification reaction is a polymerase chain reaction (PCR).

3. The method of claim 2, wherein said determining is conducted at at least two cycles of PCR.

4. The method of claim 2, wherein said determining is conducted at each of the PCR cycles.

5. The method of claim 2, wherein said determining is conducted at every second of the PCR cycles.

6. The method of claim 2, wherein said determining is conducted at every third of the PCR cycles.

7. The method of claim 1, wherein the oligonucleotides further comprise oligonucleotide primers and said determining further comprises determining change in mass of the target DNA and the oligonucleotide primers.

8. The method of claim 1, wherein the target DNA becomes bound to a surface, and said determining comprises determining the change in the mass of the target DNA bound to the surface.

9. The method of claim 8, wherein binding of the target DNA to the surface is reversible.

10. The method of claim 8, wherein reactant monomeric triphosphate monomers do not bind the surface.

11. The method of claim 8, further comprising determining temperature at which the oligonucleotides become unbound from the surface, wherein said determining temperature is conducted after the amplification reaction.

12. The method of claim 1, wherein said determining is conducted in situ.

13. The method of claim 1, wherein said determining is conducted via a mass-sensitive detector.

14. The method of claim 13, wherein the mass-sensitive detector is selected from the group consisting of a white light interferometer, a surface wave sensor, a microbalance, a quartz crystal microbalance dissipation sensor and a sensor for detection of surface plasmon resonance.

15. The method of claim 1, wherein the amplification reaction comprises at least one cycle of steps:
a) denaturing of double stranded template DNA at a denaturation temperature, producing single stranded template DNA;
b) annealing of the oligonucleotide primers to the single stranded template DNA to be amplified at an annealing temperature; and
c) amplifying the single stranded template DNA at a chain elongation temperature, to produce amplified DNA, wherein the target DNA comprises the template DNA and the amplified DNA,
wherein said determining is conducted after step b), after step c), or after both steps b) and c).

16. The method of claim 15, wherein the target DNA binds to a surface and detaches from the surface at the denaturation temperature.

17. The method of claim 15, wherein said determining further comprises determining a measured value at the denaturation temperature.

18. The method of claim 15, wherein said determining is conducted after step b).

19. The method of claim 15, wherein said determining is conducted at a temperature below the annealing temperature.

20. The method of claim 19, wherein said determining is conducted at a temperature in a range of from 25° C. to 50° C.

21. The method of claim 20, wherein the range is from 30° C. to 40° C.

22. The method of claim 1, wherein the target DNA comprises amplified DNA, and wherein said method further comprises determining molecular weight of the amplified DNA on the basis of the change in mass of the target DNA.

23. The method of claim 1, further comprising determining an amplification factor on the basis of the change in mass of the target DNA.

24. The method of claim 1, further comprising determining an initial concentration of target DNA prior to the amplification reaction.

25. The method of claim 1, wherein the amplification reaction is conducted in a device comprising:
a) a reaction chamber;
b) a temperature control unit;
c) a surface suitable for binding of the oligonucleotides; and
d) a detector for determining the mass of the oligonucleotides bound on the surface.

26. A device for quantitative and/or qualitative detection of nucleic acid molecules amplified in an amplification reaction, comprising:
a) a reaction chamber;
b) a temperature control unit;
c) a surface that binds oligonucleotides comprising amplified nucleic acid molecules and/or oligonucleotide primers; and
d) a detector for determining mass of the oligonucleotides bound on the surface.

27. The device of claim 26, wherein said surface binds the oligonucleotides unspecifically and/or with similar affinity.

28. The device of claim 26, wherein said surface preferentially binds the amplified nucleic acid molecules compared to the oligonucleotide primers.

29. The device of claim 26, wherein said surface does not bind deoxyribnucleoside triphosphates.

30. The device of claim 26, wherein said surface comprises binding sites for the oligonucleotides.

31. The device of claim 30, wherein said binding sites are evenly distributed on said surface.

32. The device of claim 30, wherein said binding sites comprise oligonucleotide probes attached to the surface, and which hybridise with the amplified nucleic acid molecules and/or the oligonucleotide primers.

33. The device of claim 32, wherein said oligonucleotide probes have the same length.

34. The device of claim 32, wherein said oligonucleotide probes are produced combinatorially on said surface.

35. The device of claim 26, wherein said surface comprises silanized glass.

36. The device of claim 26, wherein said surface has a thickness suitable for interferometry.

37. The device of claim 36, wherein the thickness is in the range of from 300 nm to 700 nm.

38. The device of claim 36, wherein the thickness is in the range of from 400 nm to 600 nm.

39. The device of claim 36, wherein the thickness is about 500 nm.

40. The device of claim 30, wherein number of said binding sites exceeds number of the oligonucleotides.

41. The device of claim 26, wherein ratio of said surface to chamber volume is from 10⁻¹ mm²/μl to 10⁻³ mm²/μl.

42. The device of claim 41, wherein the ratio is from 5×10⁻² mm² to 5×10⁻³ mm²/μl.

43. The device of claim 41, wherein the ratio is about 0.02 mm²/μl.

44. The device of claim 26, further comprising (e) a control computer for recording data and controlling temperature and said detector.

45. The device of claim 26, further comprising (f) a sample inlet and (g) a sample outlet.

46. The device of claim 26, wherein said detector is selected from the group consisting of a white light interferometer, a surface wave sensor, a microbalance, a quartz crystal microbalance dissipation sensor and a sensor for detection of surface plasmon resonance.
